(19) United States
(12) Patent Application Publication
(10) Pub. No.: US 2004/0162508 A1
(43) Pub. Date: Aug. 19, 2004

(54) SHOCK WAVE THERAPY METHOD AND DEVICE

(76) Inventor: Walter Uebelacker, Kennesaw, GA (US)

Correspondence Address: SEYFARTH SHAW, 55 EAST MONROE STREET, SUITE 4200, CHICAGO, IL, 60603-5803, US

(21) Appl. No.: 10/708,249

(22) Filed: Feb. 19, 2004

Related U.S. Application Data
(60) Provisional application No. 60/448,981, filed on Feb. 19, 2003.
(60) Provisional application No. 60/448,979, filed on Feb. 19, 2003.

(51) Int. Cl. A61B 17/22

(52) U.S. Cl. 601/2; 601/4

(57) ABSTRACT

An extracorporeal shock wave system provides a planar wave for the treatment of tissue. A parabolic reflector is provided in order to propagate the planar wave through a membrane and to the tissue of a human subject. A piezoelectric, electrohydraulic or electromagnetic source may be used to develop the wave.

### CROSS REFERENCE TO RELATED APPLICATIONS

The present application claims the benefit of the filing date of provisional application Nos. 60/448,981 and 60/448,979 both filed Feb. 19, 2003.

### BACKGROUND OF INVENTION

The present application relates to extracorporeal shock wave technology and in particular, an electromagnetic, electrohydraulic or piezoelectric shock wave device that propagates planar waves, and to methods of using such a device, for developing shock waves and for treating tissue.

Shock waves are used in different medical disciplines and in different species. Although it is not known exactly how specific tissue responds to the shock wave, it is proven that shock waves can have a therapeutic effect and improve certain medical conditions.

In urology, the shock wave is used to disintegrate kidney or urethra stones. In orthopedics, shock waves are used to stimulate bone growth in non-unions. Shock wave therapy is further used to treat epicondylitis, tendonitis calcarea of the shoulder, achillodynia calcaneal spurs, and many other conditions. Shock waves are also used in veterinary medicine to treat ligaments, tendons, splint bone fractures, navicular syndrome, back pain, and certain joint conditions.

Commercially available devices use either high-energy focused shock wave systems or radial emitting pressure pulse systems. In these systems the shock wave is generated either by an electrical discharge in a liquid (electro hydraulic), electrical discharge in an electrical coil that drives a diaphragm (electro magnetic), electrical discharge in piezo elements (piezo electric) or a projectile that hits its target (ballistic system).

Focused shock wave systems have an advantage over radial systems because the shock wave reaches its maximal density inside the body. This allows for the treatment of deeper tissue inside the body. Typical penetration depths in orthopedic devices are 100 mm in human medicine or up to 80 mm in veterinary medicine.

Radial systems can only treat superficial conditions because the diverging wave loses energy density with the square of the distance to the source, leading to insufficient energy density to show an effect on deeper tissue inside the body.

Investigations have shown that, for a tissue to respond, the shock wave must reach a certain energy density measured in mJ/mm² (milli Joules per square millimeter).

Also the volume of the treated tissue (or area for rather two-dimensional treatment regions, such as tendons) plays an important factor. Treatment results show that these two factors have the major influence on the clinical outcome.

Focused systems have enough energy density in deeper regions but the treatment area is often too small. Either the shock wave source or the patient must be moved to treat a bigger area.

Radial systems treat a bigger area, but the power density is too small to show an effect in deeper tissues.

The task of the present invention is to optimize the interaction of the shock wave with the tissue of a subject being treated so as to achieve the best clinical result. This task is accomplished by using high-energy shock waves that are generated by electro hydraulic, electro magnetic, or piezoelectric means, but not focused into a focal point. Instead, the shock wave is reflected or refracted in such a way that a “plane wave” or “flat wave” is emitted from the source.

With a “plane” or “flat” wave, the energy is neither converging (as with the focused shock wave) or diverging (as with a radial wave). Rather the energy distribution over the emitting area stays the same even in different distances along the axis of the shock wave source. The initial shock wave energy must be enough to reach a certain energy density at the distal end of the shock wave source.

FIG. 1 shows a drawing of a conventional device having a high-voltage generator that stores electrical energy in capacitors. Electrode tips 2 and 3 are electrically connected to the high-voltage unit 7 and are disposed in a housing 1 for a reflector 4. The housing 1 is filled with a liquid W. In a preferred embodiment, the liquid W is water. To keep the water within the housing, it is sealed by a membrane M. A spark is generated between the two electrode tips 2 and 3 which are centered at the focal point F1 of the ellipsoid, to generate a shock wave 8. The membrane M provides a contact surface of the device to the treatment area. As the shock wave is expanding it will hit the reflector of an ellipsoidal shape. The inner surface of reflector 4 has an ellipsoid shape to reflect the shock wave, as at 10 and 10″, toward focal point F2. The reflected part of the spherical shockwave represented by the space angle e is determined by the cutoff point (M) of the ellipsoid and by the half axes of a and b of the ellipsoid.

### SUMMARY OF INVENTION

The present invention pertains to a shock wave device comprising a reflector housing, a parabolic reflector disposed in the housing, and an energy source disposed within the reflector for developing a shock wave so that a planar shock wave is formed by the reflector and emanates from the housing. In an embodiment, the reflector is shaped and dimensioned to provide a reflected wave having a power density level to produce a tissue reaction in a subject to which the wave is administered. In an embodiment, the power density may be in the range of approximately 0.01 mJ/mm² to 1.0 mJ/mm². In an embodiment, the opening of the paraboloid may have a diameter in the range of approximately 20 mm to 100 mm. In an embodiment, the distance between the origin point of the paraboloid to a propagation point may be in the range of approximately 3 mm to 10 mm.

In an embodiment, the energy source may be an electro hydraulic source. In an embodiment, the energy source may have a propagation point centered approximately at the focal point of the parabolic reflector. In an embodiment, the energy source may comprise a pair of electrode tips connected to a capacitor. In an embodiment, the energy source may have a propagation point centered approximately between the electrode tips. In an embodiment, the reflector may include a cavity having an opening sealed by a membrane. In an embodiment, the cavity may contain a fluid. In an embodiment, the fluid may be water.

An embodiment of the invention may provide for a method for developing a planar shock wave to be used for therapeutic purposes on a subject, the method comprising the steps of generating a spark to cause a shock wave, shaping and directing the shock wave to create a planar wave and propagating the planar shock wave toward the subject. In an embodiment, the method may further comprise the steps of providing a device having a parabolic reflector, an energy source attached to an electrode tip and a membrane disposed across a cavity in communication with the parabolic reflector, orienting the electrode tip at a focal point of the parabolic reflector, generating a spark at the electrode tip and developing a shock wave, propagating the shock wave so that it reflects at the parabolic reflector, forming a planar wave, propagating the planar wave through the membrane and toward tissue of a subject to receive the planar wave for therapeutic effect.

An embodiment of the invention provides a method for treating tissue comprising the steps of generating a planar shock wave and coupling the planar shock wave to the tissue to be treated. In an embodiment, the method may further comprise the steps of providing a treatment device that develops a shock wave, orienting the treatment device adjacent to the tissue area, forming a planar shock wave to be propagated from the treatment device and to be dispersed through the tissue and activating the tissue in order to cause a chemical release from the tissue cells. In an embodiment, the shockwave may be developed by electro hydraulic, electromagnetic or piezoelectric means. In an embodiment, the method may comprise the steps of generating a spark by an electrode tip to develop the shockwave and reflecting the shockwave from a parabolic reflector. In an embodiment, the tissue is activated to release a protein for generating an immune response.

An embodiment of the invention provides for a therapeutic device for administering a shock wave to a subject comprising a housing, a shock wave source disposed in the housing, wave directing and shaping structure in the housing responsive to the shock wave for causing a planar shock wave to be emitted from the housing, and structure for coupling the shock wave to the subject. In and embodiment the wave directing and shaping structure includes a parabolic reflector. In an embodiment the housing includes an opening and the coupling structure includes a membrane disposed across the opening. In an embodiment the wave directing and shaping structure is disposed in a cavity having the opening. In an embodiment the shock wave source includes an electrode that develops a spark.

### BRIEF DESCRIPTION OF DRAWINGS

For the purpose of facilitating an understanding of the invention, there is illustrated in the accompanying drawings an embodiment thereof, from an inspection of which, when considered in connection with the following description, its construction and operation, and many of its advantages should be readily understood and appreciated.

FIG. 1 is a diagrammatic view of a section through a prior art shock wave device propagating a focused wave; and

FIG. 2 is a view similar to FIG. 1 of a shock wave device of the present invention propagating a planar wave.

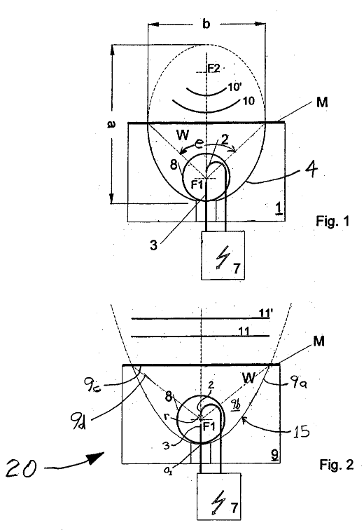

### DETAILED DESCRIPTION

FIG. 2 depicts a device 20 of the present invention including the high-voltage generator 7 that stores electrical energy in capacitors and is provided with the electrodes 2 and 3. The amount of electrical energy is given by the voltage and the capacitance and usually the capacitors are charged to 10 kV to 30 kV, the capacitance being in the range from 10 nF to 50 nF, leading to electrical energy stored in the capacitors in the range of from 0.5 J to 23 J for an electrohydraulic system.

A reflector housing in an embodiment may be made of ceramic, brass, steel, aluminum or other metals. In an embodiment, the housing 9 is cylindrically shaped. In the housing 9 is a reflector 15 which has a parabolic shape (as shown in Fig. In an embodiment, the reflector 15 and housing 9 may be integrally formed. In an alternate embodiment, the reflector 15 may be a separate surface from the housing 9 and a wall 9a of the reflector 15 has a thickness of approximately 3 mm. The reflector housing 9 includes a cavity 9b that is filled with a fluid W that transmits the shockwave. In an embodiment the fluid W is water. To keep the water contained within the cavity 9b, the housing 9 is sealed by a membrane M. In an embodiment, the membrane M consists of soft PVC and its wall thickness is in the range of approximately 1 to 3 mm. PVC has a good acoustic matching to the water so that the reflection losses will be low. The membrane M may also provide a contact surface of the device to the treatment area. To achieve a good acoustic coupling of the shock wave from the device into the treatment area a coupling gel, such as ultra sound gel, may be used.

The device 20 includes a wave directing and shaping structure, such as the reflector wall that is formed having a parabolic shape. Water is contained within the paraboloid The paraboloid has an origin O₁ and focal point In a preferred embodiment, the distance between F1 and O₁ is approximately 3 mm to 10 mm.

In use, a high-voltage discharge from the capacitor 7 causes a spark to be generated between the electrode tips 3 and 2, which are disposed substantially at the focal point The spark provides a shock wave source that creates a spherical shock wave illustrated as a circle The wave is illustrated in FIG. prior to reflection. In an electro hydraulic system, the shock wave 8 generates a plasma bubble. The focal point F1 provides a propagation point that is centered between the electrode tips and 3.

As the plasma bubble expands spherically and cools down, it drives a shock wave in front of it. If the expansion velocity of the plasma bubble is lower than the velocity of sound of the surrounding medium W, a spherical shock wave is released and detaches from the expanding plasma bubble. As the wave propagates, its lower portion will reflect against the lower portion of the parabolic reflector and propagate a planar wave that will move through the reflector cavity The planar wave will move toward the opening of the cavity which is defined by the intersection with the membrane M of a conical angle r having its apex at the focal point F1. The wave then propagates through the membrane that couples the shock wave and will propagate it through the skin and tissue of the subject which the membrane is placed against.

The energy density of the shock wave is determined for a given energy by the distance of F1 from the origin point O₁ of the paraboloid. The reflected part of the spherical shockwave represented by the space angle r is determined by the cut-off distance (M) of the paraboloid from its focal point. The wave propagates in a way that a flat shock wave and 11″ is released from the shock wave device. The wave propagates into the patient as represented by wave and a wave further in time”. In a preferred embodiment, the paraboloid has an opening 9c having a diameter which is in the range of approximately 20 mm to 100 mm. In a preferred embodiment, the power density of the wave is in the range of 0.01 mJ/mm² to 1 mJ/mm².

In an alternate embodiment, the device 20 may be piezo electric or electromagnetic and provide a wave via means other than the electrohydraulic system depicted in Fig. In such embodiments, a lens may be used in place of the reflector 15. In a further alternate embodiment, a rod which forms a cylindrical wave source wave may be used. In such an embodiment, the reflector may have side walls forming a conical angle of approximately 45° in order to develop the planar wave.

The above arrangement depicted in FIG. wherein F1 is approximately 3 mm to 10 mm from the origin of the paraboloid, will provide a wave that has a proper power density so that the wave can affect tissues in a human body in order to cause a therapeutic effect. For example, an energy density is high enough to trigger a physiological repair response within the cell. Such mechanisms may include release of cytokines induction of heat, shock, protein and other immunological responses. Such responses may be generated by a planar shock wave of 50 to 1,000 isonorm bars. This planar wave will penetrate deeply into a human subject so that tissue treatments may be helpful through a large area.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants” contribution. The actual scope of the protection sought is intended to be defined in the following claims when viewed in their proper perspective based on the prior art.

1. A therapeutic shock wave device comprising:
a reflector housing;
a parabolic reflector disposed in the housing; and
an energy source disposed within the parabolic reflector for developing a shock wave so that a planar shock wave is formed by the parabolic reflector and emanates from the housing.

2. The device of claim 1 wherein the parabolic reflector is shaped and dimensioned to provide the planar shock wave having a power density level to produce a tissue reaction in a subject to which the wave is administered.

3. The device of claim 1 wherein the shock wave has a power density in the range of approximately 0.01 mJ/ mm² to 1.0 mJ/mm².

4. The device of claim 1, and further comprising a coupling member which intersects the reflector along a circle having a diameter in the range of approximately 20 mm to 100 mm.

5. The device of claim 1 wherein the parabolic reflector has an origin point and a focal point spaced from the origin point a distance in the range of approximately 3 mm to 10 mm.

6. The device of claim 1 wherein the energy source is an electrohydraulic source.

7. The device of claim 1 wherein the energy source has a propagation point centered approximately at a focal point of the parabolic reflector.

8. The device of claim 1 wherein the energy source comprises a pair of electrode tips connected to a capacitor.

9. The device of claim 8 wherein the energy source has a propagation point centered approximately between the electrode tips.

10. The device of claim 1 wherein the parabolic reflector includes a cavity having an opening and the opening sealed by a membrane.

11. The device of claim 9 wherein the cavity contains a fluid.

12. The device of claim 10 wherein the fluid is water.

13. A method for developing a planar shock wave to be used for therapeutic purposes on a subject, the method comprising the steps of:
generating a spark to cause a shock wave;
shaping and directing the shock wave to create a planar shock wave; and
propagating the planar shock wave toward the subject.

14. The method of claim 13 further comprising the steps of:
providing a device having a parabolic reflector, an energy source attached to an electrode tip and a membrane disposed across a cavity in communication with the parabolic reflector;
orienting the electrode tip generally at a focal point of the parabolic reflector;
generating the spark at the electrode tip and developing the shock wave;
propagating the shock wave so that it reflects at the parabolic reflector; and
propagating the planar shock wave through the membrane and toward tissue of the subject to receive the planar wave for therapeutic effect.

15. The method of claim 13 wherein the planar shock wave generates an immune response in the subject and has a power density in the range of approximately 0.01 mJ/ mm² to 1.0 mJ/mm².

16. The method of claim 14 wherein the parabolic reflector has an opening having a diameter that is in the range of approximately 20 mm to 100 mm.

17. The method of claim 14 wherein the planar shock wave triggers a physiological repair response in the subject.

18. A therapeutic method for treating tissue comprising the steps of:
generating a planar shock wave; and
coupling the planar shock wave to the tissue to be treated.

19. The method of claim 18 further comprising the steps of:
providing a treatment device that develops the planar shock wave;
orienting the treatment device adjacent to the tissue area; and
activating the tissue in order to cause a chemical release from the tissue cells.

20. The method of claim 18 wherein the shock wave is generated by electro hydraulic, electromagnetic or piezoelectric means.

21. The method of claim 18 wherein the generating includes:
generating a spark to develop a shockwave, and reflecting the shockwave from a parabolic reflector to form a planar shock wave.

22. The method of claim 18 wherein the planar shock wave is administered at a power density sufficient to cause the tissue to be activated to release a protein for generating an immune response.

23. A therapeutic device for administering a shock wave to a subject comprising:
a housing;
a shock wave source disposed in the housing;
wave directing and shaping structure in the housing responsive to the shock wave for causing a planar shock wave to be emitted from the housing; and
structure for coupling the shock wave to the subject.

24. The therapeutic device of claim 23 wherein the wave directing and shaping structure includes a parabolic reflector.

25. The therapeutic device of claim 23 wherein the housing includes an opening and the coupling structure includes a membrane disposed across the opening.

26. The therapeutic device of claim 25 wherein the wave directing and shaping structure is disposed in a cavity having the opening.

27. The therapeutic device of claim 23 wherein the shock wave source includes an electrode that develops a spark.
